(19) United States
(12) Patent Application Publication
(10) Pub. No.: US 2004/0248972 A1
(43) Pub. Date: Dec. 9, 2004

(54) Compounds and uses thereof

(75) Inventors: David J. Lockhart, Del Mar, CA (US); Hitesh K. Patel, Encinitas, CA (US); Zdravko V. Milanov, San Diego, CA (US); Shamal Anil Mehta, San Diego, CA (US)

Correspondence Address: WILSON SONSINI GOODRICH & ROSATI, 650 PAGE MILL ROAD, PALO ALTO, CA, 943041050

(73) Assignee: Ambit Biosciences Corporation, San Diego, CA

(21) Appl. No.: 10/848,515

(22) Filed: May 17, 2004

Related U.S. Application Data
(60) Provisional application No. 60/471,425, filed on May 16, 2003.
(60) Provisional application No. 60/480,289, filed on Jun. 20, 2003.
(60) Provisional application No. 60/488,178, filed on Jul. 16, 2003.
(60) Provisional application No. 60/488,172, filed on Jul. 16, 2003.
(60) Provisional application No. 60/480,475, filed on Jun. 20, 2003.
(60) Provisional application No. 60/516,610, filed on Oct. 30, 2003.
(60) Provisional application No. 60/516,651, filed on Oct. 30, 2003.
(60) Provisional application No. 60/516,616, filed on Oct. 30, 2003.

(51) Int. Cl. A61K 31/366; A61K 31/225

(52) U.S. Cl. 514/460; 514/548

(57) ABSTRACT

The invention provides derivatives of statin compounds, preferably with reduced, or unappreciable, inhibitory activity against HMG-CoA reductase as well as compositions comprising such compounds. The invention also provides methods of using the compounds and/or compositions in the treatment of a variety of diseases and unwanted conditions in subjects. Kits comprising the non-statin compounds of the invention are also provided.

### CROSS-REFERENCE TO RELATED APPLICATIONS

This application claims the benefit of U.S. Provisional Application No. 60/471,425 filed May 16, 2003, U.S. Provisional Application No. 60/480,289 filed Jun. 20, 2003, U.S. Provisional Application No. 60/488,178 filed Jul. 16, 2003, U.S. Provisional Application No. 60/488,172 filed Jul. 16, 2003, U.S. Provisional Application No. 60/480,475 filed Jun. 20, 2003, U.S. Provisional Application No. 60/516,610 filed Oct. 30, 2003, U.S. Provisional Application No. 60/516,651 filed Oct. 30, 2003 and U.S. Provisional Application No. 60/516,616 filed Oct. 30, 2003 all of which are incorporated herein by reference.

### BACKGROUND

The statin compounds, which include lovastatin, simvastatin, pravastatin, fluvastatin, atorvastatin (also known as Lipitor™), and cerivastatin have been characterized and used in relation to the inhibition of HMG-CoA reductase activity.. The conversion of 3-hydroxy-3-methylglutaryl coenzyme A to mevalonate is catalyzed by HMG-CoA reductase. This is an early and rate-limiting step in cholesterol synthesis. Inhibition of HMG-CoA reductase decreases intracellular cholesterol, which leads to an up regulation of LDL-receptors and the more rapid clearance of LDL-cholesterol from plasma. Cholesterol is an essential component in VLDL particle assembly and release, so lowered cholesterol levels would also decrease plasma triglycerides. The statin compounds have been used in relation to lowering cholesterol as well as low density lipoprotein (LDL) and triglyceride levels in subjects in need of such lowering. In addition to HMG CoA reductase, it is possible that the statins target other cellular constitiuents. Hence, it would be desirable to identify additional targets of statins and also synthesize additional statin derivatives that exhibit similar or different properties from the known statins.

The above is not intended as an admission that any of the foregoing is pertinent prior art. All statements as to the date or representation as to the contents of documents herein is based on the information available to the applicant and does not constitute any admission as to the correctness of the dates or contents of these documents.

### SUMMARY OF THE INVENTION

The present invention is directed to non-statin compounds and uses thereof. The non-statin compounds comprise of statin compounds lacking the moiety that interacts with HMG CoA reductase. These non-statin compounds are preferably used in conditions wherein an effect on HMG CoA reductase is not desired, preferably in conditions wherein an effect on PDE6 is desired. In one aspect, the statin compounds are used in the treatment of PDE6-related conditions.

### DETAILED DESCRIPTION OF THE INVENTION

The present invention is directed to derivatives of statin compounds and methods for their use as drugs. The non-statin compounds disclosed herein comprise a portion of a statin compound that lacks the moiety believed to be involved in inhibitory activity against 3-hydroxy-3-methylgluatryl-coenzyme A (HMG-CoA) reductase. Preferably, the non-statin compounds of the present invention lack the lactone ring, the heptanoic acid, or the protected form of the heptanoic acid.

The compounds are derivatives of two subclasses of statin compounds. The first subclass is the hexahydronaphthalene subclass (having a hexahydronaphthalene ring in common) as represented by lovastatin (also known as Altocor and Mevacor), simvastatin (Zocor), and pravastatin (Pravachol). The second subclass is the fluorophenyl subclass, having a fluorophenyl ring in common, as represented by fluvastatin (Lescol), atorvastatin (Lipitor), Rosuvastatin (Crestor), pitavastatin, dalvastatin, glenvastatin, bervastatin, and cerivastatin (Baycol™). An additional statin is carvastatin.

The non-statin compounds of the invention may be used to treat a variety of diseases and unwanted conditions, including, but not limited to, cerebral accident (or cerebrovascular accident, including stroke), inflammation (including inflammation due to autoimmune diseases), multiple sclerosis, blood vessel growth (angiogenesis), bone formation/bone growth, immune system stimulation, acute coronary syndromes (including myocardial infarction, non-Q-wave myocardial infarction and unstable angina), and cardiovascular disease. In one aspect, the compounds of the invention may be used to reduce the likelihood of stroke or cardiovascular disease, and to decrease damage following brain and/or heart infarction or other trauma. In another aspect, the compounds may be used to reduce the severity or damage caused by stroke or cardiovascular disease in a subject. Non-limiting examples of the benefit provided by the compounds include decreased brain and/or heart infarction.

Compounds

The non-statin compounds of the invention include those represented by the following formula I:

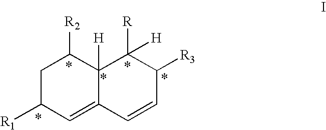

The invention also provides for isomeric compounds based on the stereochemistry of the hydronaphthyl portion of the molecule represented above to result in compounds based upon a decalin structure which contains at least one double bond. Additionally, the compounds may be analogs of such decalin structures where the double bond is hydrogenated or cyclopropylated at any permitted position in the decalin structure (with one such position exemplified by I′″″ below). Non-limiting examples of such compounds are represented by the following formulae as follows:

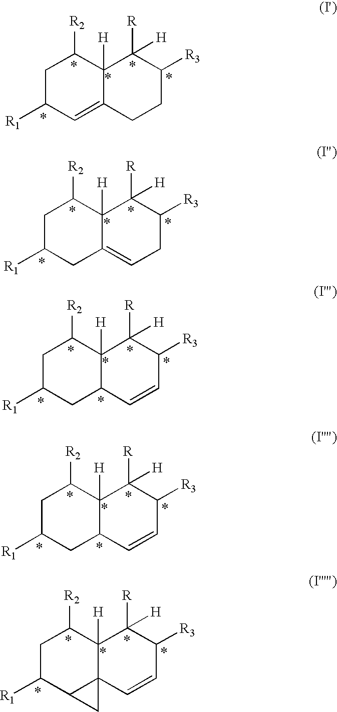

wherein the carbon atom positions denoted by “*” are chiral atoms and all possible stereoisomers, as well as racemic mixtures thereof, are provided by the invention; and wherein R₁, R₂ and R₃ are independently selected from H, OH, or acylated OH as described below; any straight or branched, saturated or unsaturated aliphatic C1-C10 chain, optionally substituted with single or multiple hydroxyl groups on the chain; and R is H or OH; a straight or branched C1-10 saturated or unsaturated aliphatic (including methyl), carboxyalkyl, carbalkoxyalkyl, or. alkoxy; a C1-8 alicyclic; a single or multiring aromatic substituted aliphatic; an aliphatic-substituted single or multiring aromatic; a single or multiring aromatic heterocycle; a single or multiring heterocyclic aliphatic; a sulfonyl; or cyano; or a naturally or non-naturally occurring amino acid. Alternatively, R is benzyl or the lactone form, or a bioisostere of the corresponding position in the statin molecule corresponding to the formulae disclosed by the present invention.

Bioisosterism refers to the use of substituents or groups that are chemically or physically similar to an existing group or moiety. Therefore, R may be

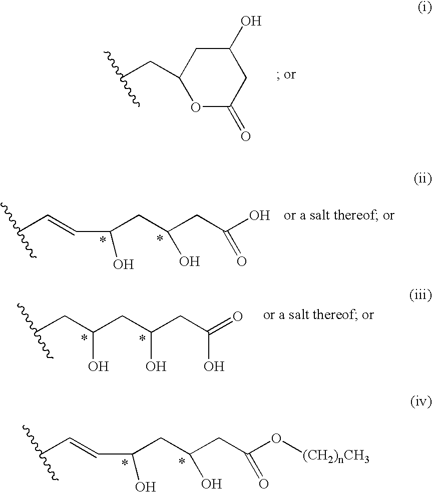

wherein n is an integer from 1-6; or any of moieties (ii) or (iii) above wherein the terminal carboxylic acid group (—COOH) is substituted by

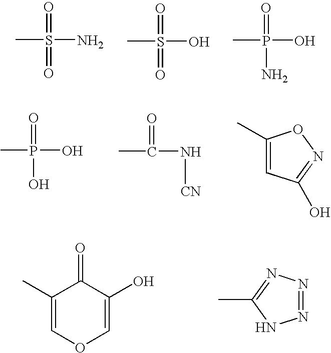

In certain embodiments, R is not one of the following:

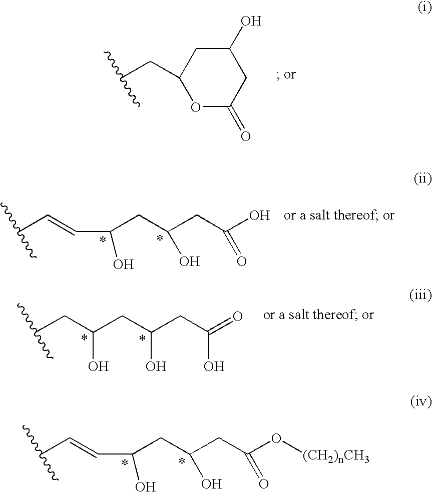

wherein n is an integer from 1-6; or any of moieties (ii) or (iii) above wherein the terminal carboxylic acid group (—COOH) is substituted by

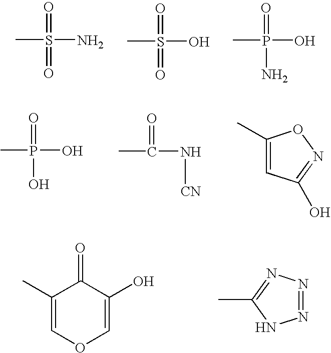

R₁, R₂ and R₃ may also be linked to the rest of the above formulae by a group independently selected from the following

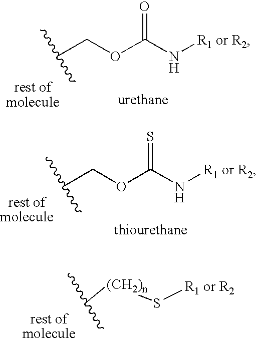

wherein n is an integer from 0 to 6, or

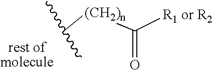

Where chiral centers occur in the compounds having the R, R₁, R₂ and R₃ moieties as defined above, the invention includes the enantiomeric compounds resulting from the chiral center as well as racemic mixtures thereof.

As used herein, aliphatic includes alkanes, olefins (alkenes or alkyldienes), and alkynes. In particular, R may be a C1-10, C2-C9, C3-C8, C4-C7, C5-C6 alkyl, alkene or alkyldiene. Preferably, R is a lower alkyl group, such as methyl, ethyl, n-propyl, isopropyl, t-butyl and n-pentyl; a lower alkyl carboxylic acid, such as formyl, carboxymethyl, carboxyethyl, carboxy-n-butyl, carboxy-sec-butyl, carboxy-n-hexyl; —(CH₂)ₙOH or —(CH₂)ₙCOO⁻ or an ester thereof represented by —(CH₂)ₙCOO(CH₂)ₙCH₃, wherein each occurrence of n is independently an integer from 1-6 and each CH₂ position may be optionally substituted by OH, fluoro, chloro, bromo or iodo. Non-limiting examples of R include —CH₂CH₂COOCH₃, —CH₂CH₂COOCH₂CH₃, —CH₂CH(CH₃)COOCH₂CH₃, —CH₂CH₂CH₂COOCH₂CH₂CH₃, —CH₂CH(CH₃)₂COOCH₂CH₃.

Alicyclic includes cycloparaffins (saturated), cycloolefins (unsaturated with two or more double bonds), and cycloacetylenes (cyclynes) with at least one triple bond. Non-limiting examples include cyclopropane, cyclohexane, cyclopentane, cyclopentadiene, and cycloctatetraene. Aromatic refers to unsaturated cyclic hydrocarbons of one or more rings and includes aryl structures typified, but not limited to, phenyl, naphthalyl, phenanthrenyl, and anthracenyl. Non-limiting aromatic examples include 6 membered (typified by benzene) as well as 5 membered (typified by furan, thiophene, pyrrole, and indole) rings. Heterocycle refers to the presence of at least one non-carbon atom in a cyclic structure. Non-limiting examples include the presence of a nitrogen, oxygen, and sulfur atom to result in heterocyclic rings including, but not limited to, phenyl, naphthyl, pyridinyl, pyrimidinyl, pyrazinyl, pyridazinyl, pyrrolyl, imidazolyl, pyrazolyl, thienyl, faryl, tetrahydrofuryl, isoxazolyl, isothiazolyl, quinolinyl, isoquinolinyl, indolyl, benzimidazolyl, benzofuranyl, benzoxazolyl, benzisoxazolyl, benzpyrazolyl, benzothiofuranyl, cinnolinyl, pterindinyl, phthalazinyl, naphthypyridinyl, quinoxalinyl, quinazolinyl, purinyl andindazolyl; wherein such phenyl, naphthyl or heterocyclic group is optionally substituted with one to five groups selected from the group consisting of a C1-6 branched or unbranched alkyl, phenyl, naphthyl, heterocycle selected from the group hereinabove described, C1-6 branched or unbranched alkyl which is optionally partially or fully halogenated, cyclopropyl, cyclobutyl, cyclopentanyl, cyclohexanyl, cycloheptanyl, bicyclopentanyl, bicyclohexanyl, bicycloheptanyl, phenyl C1-5 alkyl, naphthyl C1-5 alkyl, halo, hydroxy, cyano, C1-3 alkyloxy which may optionally be partially or fully halogenated, phenyloxy, naphthyloxy, heteraryloxy wherein the heterocyclic moiety is selected from the group hereinabove described, nitro, amino, mono- or di-(C1-3)alkylamino, phenylamino, naphthylamino, heterocyclylamino wherein the heterocyclyl moiety is selected from the group hereinabove described, NH₂C(O), a mono- ordi-(C1-3)alkyl aminocarbonyl, C1-5 alkyl-C(O)-C1-4 alkyl, amino-C1-5 alkyl, mono-or di-(C1-3)alkylamino-C1-5 alkyl, amino-S(O)2, or di-(C1-3)alkylamino-S(O)2; or a fused aryl selected from the group consisting of benzocyclobutanyl, indanyl, indenyl, dihydronaphthyl, tetrahydronaphthyl, benzocycloheptanyl and benzocycloheptenyl, or a fused heterocyclyl selected from the group consisting of cyclopentenopyridine, cyclohexanopyridine, cyclopentanopyrimidine, cyclohexanopyrimidine, cyclopentanopyrazine, cyclohexanopyrazine, cyclopentanopyridazine, cyclohexanopyridazine, cyclopentanoquinoline, cyclohexanoquinoline, cyclopentanoisoquinoline, cyclohexanoisoquinoline, cyclopentanoindole, cyclohexanoindole, cyclopentanobenzimidazole, cyclohexanobenzimidazole, cyclopentanobenzoxazole, cyclohexanobenzoxazole, cyclopentanoitnidazole, cyclohexanoimidazole, cyclopentanothiophene and cyclohexanothiophene, wherein the fused aryl or fused heterocyclyl ring issubstituted with 0 to 3 groups independently selected from phenyl, naphthyl and heterocyclyl selected from the group consisting of pyridinyl, pyrimidinyl,pyrazinyl, pyridazinyl, pyrrolyl, imidazolyl, pyrazolyl, thienyl, furyl, isoxazolyl, and isothiazolyl, C1-6 branched or unbranched alkyl which is optionally partially or fully halogenated, halo, cyano, C1-3 alkyloxy which is optionally partially or fully halogenated, phenyloxy, naphthyloxy, heterocyclyloxy wherein the heterocyclyl moiety is selected from the group hereinabove described, nitro, amino, mono- or di-(C1-3)alkylamino, phenylamino, naphthylarnino, heterocyclylamino wherein the heterocyclylmoiety is selected from the group hereinabove described, NH₂C(O), a mono- or di-(C1-3)alkyl aminocarbonyl, C1-4 alkyl-OC(O), C1-5 alkyl-C(O)-C1-4 branched or unbranched alkyl, an amino-C1-5 alkyl, or mono- or di-(C1-3)alkylamino-C1-5 alkyl; or c) cycloalkyl selected from the group consisting of cyclopentanyl, cyclohexanyl, cycloheptanyl, bicyclopentanyl, bicyclohexanyl and bicycloheptanyl, wherein the cycloalkyl may optionally be partially or fully halogenated and which may optionally be substituted with one to three C1-3 alkyl groups; or azetidinyl, pyrrolidinyl, piperidinyl, morpholino, piperazinyl, hexahydroazepinyl or octahydroazocinyl.

All of the above described aliphatic, carboxyalkyl, carbalkoxyalkyl, alkoxy, alicyclic, aryl, aromatic, and heterocyclic moieties may, of course, also be optionally substituted with 1-3 substituents independently selected from halo (fluoro, chloro, bromo or iodo), lower alkyl (1-6C) and lower alkoxy (1-6C).

As used herein, carboxyalkyl refers to the substituent —R′—COOH wherein R′ is alkylene; and carbalkoxyalkyl refers to —R′—COOR wherein R′ and R are alkylene and alkyl respectively. In preferred embodiments, alkyl refers to a saturated straight- or branched-chain hydrocarbyl radical of 1-6 carbon atoms such as methyl, ethyl, n-propyl, isopropyl, n-butyl, t-butyl, n-pentyl, 2-methylpentyl, n-hexyl, and so forth. Alkylene is the same as alkyl except that the group is divalent.

Sulfonyl refers to the presence of a sulfur atom, which is optionally linked to another moiety such as an aliphatic group, an aromatic group, an aryl group, an alicyclic group, or a heterocyclic group. Aryl or alkyl sulfonyl moieties have the formula —SO₂R′, and alkoxy moieties have the formula —O—R′, wherein R′ is alkyl, as defined above, or is aryl wherein aryl is phenyl, optionally substituted with 1-3 substituents independently selected from halo (fluoro, chloro, bromo or iodo), lower alkyl (1-6C) and lower alkoxy (1-6C).

The R₁ position of formulas I, I′, I″, I′″, and I″″ above may be as defined for the R position in formulas (I) and (II) of U.S. Patent Re. 36,481 in column 4, lines 1-38 therein. Definitions that define that R group are as further detailed in that Patent.

The R₂ moieties resulting from acylation of a hydroxyl group may be represented by R₂′ COO— wherein R₂′ is selected from C₁-₁₀ straight, or branched chain alkyl, C₃₋₁₀ cycloalkyl, C₂₋₁₀ alkenyl, C₁₋₁₀CF₃-substituted alkyl, phenyl, halophenyl, wherein halo is chloro, fluoro, bromo or iodo, phenyl-C₁₋₃ alkyl, substituted phenyl-C₁₋₃ alkyl in which the substituent is halo, such as fluoro, chloro, bromo, or iodo, C₁₋₃ alkyl or C₁₋₃ alkoxy. In some embodiments of the invention, R₂′ is C₂₋₅ straight chain alkyl, C₃₋₁₀ branched chain alkyl, C₃₋₁₀ cycloalkyl, and C₃₋₁₀ alkenyl in which the unsaturation is not in conjugation with the carbonyl. R₂′ may also be 1,1-diethylpropyl or 1-ethyl-1-methylpropyl. Acylation reactions for the introduction of such moieties are provided in U.S. Pat. No. 4,444,784. The R₂ position may also be the result of acylation to introduce the R—COO— structures in column 16, line 14 to column 26, line 6 (especially Tables II through VII inclusive) of U.S. Pat. No. 4,444,784.

In a preferred embodiment of the invention, R₂ is such that the resultant compound may be represented by formula II:

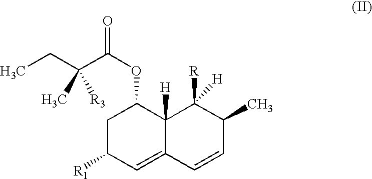

wherein R and R₁ are as described above and R₃ is H or CH₃.

In another aspect of the invention, non-statin compounds represented by the following formula III are provided

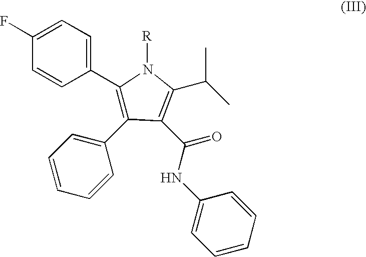

wherein R is as defined above for formula I.

The invention also provides derivative compounds based upon the core structure as depicted above and as represented by the following formula

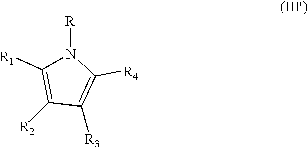

wherein R is as defined for formula I and wherein R₁ is 1-naphthyl; 2-naphthyl; cyclohexyl; norbomenyl; 2-, 3-, or 4-pyridinyl; phenyl or benzyl; phenyl or benzyl substituted with fluorine, chlorine, bromine, iodine, or hydroxyl; trifluoromethyl; alkyl of from one to four carbon atoms as described herein; alkoxy of from one to four carbon atoms; a heterocyclic moiety as disclosed above; or alkanoyloxy of from two to eight carbon atoms. Where R₁ is an aromatic or heterocyclic moiety.

One of R₂ and R₃ is —CONR₅R₆ where R₅ and R₆ are independently hydrogen; alkyl of from one to six carbon atoms as described herein; 2-, 3-, or 4-pyridinyl; phenyl or benzyl; phenyl or benzyl substituted with fluorine, chlorine, bromine, iodine or cyano; trifluoromethyl; pyridine; or carboalkoxy of from three to eight carbon atoms. The other of R₂ or R₃ is hydrogen; alkyl of from one to six carbon atoms as described herein; cyclopropyl; cyclobutyl; cyclopentyl; cyclohexyl; pheny or benzyl; phenyl or benzyl substituted with fluorine, chlorine, bromine, iodine, or hydroxyl; trifluoromethyl; alkoxy of from one to four carbon atoms; or alkanoyloxy of from two to eight carbon atoms.

R₄ is alkyl of from one to six carbon atoms as described herein; isopropyl, isobutyl, isopentyl, as well as the sec-, normal, tert- or neo- forms thereof, such as n-butyl, and t-butyl; cyclopropyl, cyclobutyl, cyclopentyl or cyclohexyl; F; Br; Cl; I; trifluoromethyl or —OR′ wherein R′ is alkyl or aryl of C1-C10.

Where R₁, R₂, or R₃ is an aromatic or heterocyclic moiety, it is optionally linked to the rest of the molecule by a straight or branched C1-C10 chain.

Preferably, R₁ is phenyl substituted with fluorine, chlorine, bromine, iodine, CF₃, or hydroxyl while R₂, R₃, and R₄ are as defined for formula III′; the substitution is preferably fluorine, optionally at thepara position relative to the bond linking the phenyl group to the rest of the molecule. Alternatively, R₂ is phenyl (or —CONR₅R₆ where R₅ and R₆ are phenyl) while R₁, R₃, and R₄ are as defined for formula III′; or R₃ is phenyl (or —CONR₅R₆ where R₅ and R₆ are phenyl) while R₁, R₂, and R₄ are as defined for formula III′. In another alternative, R₄ is —CH(CH₃)₂ or —CF₃ while R₁, R₂, and R₃ are as defined for formula III′.

Reactions for the introduction of R₁, R₂, R₃, and R₄ according to formulae III and III′ are described in U.S. Pat. No. 4,684,893.

In a further aspect provided by the invention, non-statin compounds represented by the following formula IV are provided.

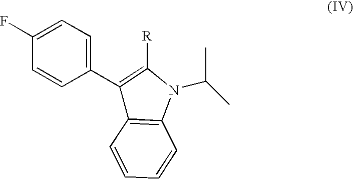

wherein R is as defined above for formula I.

The invention also provides derivative compounds based upon the core structure as depicted above and as represented by the following formula

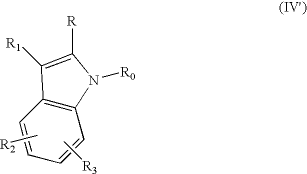

wherein R is as defined above for formula I and wherein one of R₁ and R₀ is

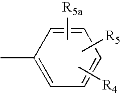

and the other is primary or secondary C₁₋₆ alkyl not containing an asymmetric carbon atom, C₃₋₆ cycloalkyl, phenyl, optionally substituted with fluorine, chlorine, bromine, iodine, CF₃, or hydroxyl, or phenyl-(CH₂)ₘ-, wherein

R₄ is hydrogen, C₁₋₃ alkyl, n-butyl, i-butyl, t-butyl, C₁₋₃ alkoxy, n-butoxy, i-butoxy, trifluoromethyl, fluoro, chloro, bromo, iodo, phenoxy or benzyloxy,

R₅ is hydrogen, C₁₋₃ alkyl, C₁₋₃ alkoxy, trifluoromethyl, fluoro, chloro, bromo, iodo, phenoxy or benzyloxy,

R₅ₐ is hydrogen, C₁₋₂ alkyl, C₁₋₂ alkoxy, fluoro or chloro, bromo, iodo, and

m is 1, 2 or 3, with the provisos that both R₅ and R₅ₐ must be hydrogen when R₄ is hydrogen, R₅ₐ must be hydrogen when R₅ is hydrogen, not more than one of R₄ and R₅ is trifluoromethyl, not more than one of R₄ and R₅ is phenoxy, and not more than one of R₄ and R₅ is benzyloxy.

Alternatively, R₀ is isopropyl, isobutyl, isopentyl, as well as the sec-, normal, tert- or neo- forms thereof, such as n-butyl, and t-butyl; cyclopropyl, cyclobutyl, cyclopentyl or cyclohexyl; F; Br; Cl; I; trifluoromethyl or —OR′ wherein R′ is alkyl or aryl of C1-C10 and R₁ is as described above.

R₂ is hydrogen, C₁₋₃ alkyl, n-butyl, i-butyl, t-butyl, C₃₋₆ cycloalkyl, C₁₋₃ alkoxy, n-butoxy, i-butoxy, trifluoromethyl, fluoro, chloro, phenoxy or benzyloxy.

R₃ is hydrogen, C₁₋₃ alkyl, C₁₋₃ alkoxy, trifluoromethyl, fluoro, chloro, phenoxy or benzyloxy, with the provisos that R₃ must be hydrogen when R₂ is hydrogen, not more than one of R₂ and R₃ is trifluoromethyl, not more than one of R₂ and R₃ is phenoxy, and not more than one of R₂ and R₃ is benzyloxy.

The R₀, R₁, R₂, R₃, and R₄ positions of formula IV′ above may be as defined for the R₀, R, R₂, R₃, and R₄ positions in the formulas of U.S. Pat. No. 5,354,772 in column 2, line 39, through column 9, line 32, and in the formulas of U.S. Pat. No. 4,739,073 in column 2, line 21, through column 8, line 37. Definitions that define those R groups are as further detailed in that Patent, which also provides reactions for the introduction of the various groups into formula IV′.

Preferably, R₀ of formula IV′ is —CH(CH₃)₂ while R₁, R₂, and R₃ are as defined in the formula. Alternatively, R₁ is a phenyl, optionally substituted with fluorine, chlorine, bromine, iodine or hydroxyl while R₀, R₂, and R₃ are as defined in formula IV′; the substitution is preferably fluorine, and preferably at the para position relative to the bond linking the phenyl group to the rest of the molecule. Alternatively, R₂ is hydrogen R₀, R₁, and R₃ are as defined for formula IV′; or R₃ is hydrogen while R₀, R₁, and R₂ are as defined for formula IV′.

In yet another aspect of the invention, non-statin compounds represented by the following formula V are provided.

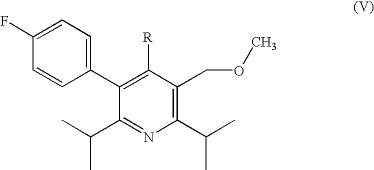

wherein R is as defined above for formula I.

The invention also provides derivative compounds based upon the core structure as depicted above and as represented by the following formula

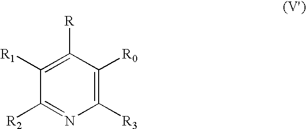

wherein R is as defined above for formula I and wherein one of R₁ and R₀ is

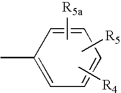

and the other is primary or secondary C₁₋₆ alkyl not containing an asymmetric carbon atom, C₃₋₆ cycloalkyl, phenyl, optionally substituted with fluorine, chlorine, bromine, iodine, CF₃, or hydroxyl, or phenyl-(CH₂)ₘ-, wherein

R4 is hydrogen, C1-3 alkyl, n-butyl, i-butyl, t-butyl, C1-3 alkoxy, n-butoxy, i-butoxy, trifluoromethyl, fluoro, chloro, bromo, iodo, phenoxy or benzyloxy,

R5 is hydrogen, C1-3 alkyl, C1-3 alkoxy, trifluoromethyl, fluoro, chloro, bromo, iodo, phenoxy or benzyloxy,

R5a is hydrogen, C1-2 alkyl, C1-2 alkoxy, fluoro or chloro, bromo, iodo, and

m is 1, 2 or 3, with the provisos that both R₅ and R₅ₐ must be hydrogen when R₄ is hydrogen, R₅ₐ must be hydrogen when R₅ is hydrogen, not more than one of R₄ and R₅ is trifluoromethyl, not more than one of R₄ and R₅ is phenoxy, and not more than one of R₄ and R₅ is benzyloxy.

Alternatively, R₀ is isopropyl, isobutyl, isopentyl, as well as the sec-, normal, tert- or neo- forms thereof, such as n-butyl, and t-butyl; cyclopropyl, cyclobutyl, cyclopentyl or cyclohexyl; F; Br; Cl; I; trifluoromethyl or —OR′ wherein R′ is alkyl or aryl of C1-C10 and R₁ is as described above.

R₂ is hydrogen, C₁₋₃ alkyl, n-butyl, i-butyl, t-butyl, C₃₋₆ cycloalkyl, C₁₋₃ alkoxy, n-butoxy, i-butoxy, trifluoromethyl, fluoro, chloro, phenoxy or benzyloxy.

R₃ is hydrogen, C₁₋₃ alkyl, C₁₋₃ alkoxy, trifluoromethyl, fluoro, chloro, phenoxy or benzyloxy, with the provisos that R₃ must be hydrogen when R₂ is hydrogen, not more than one of R₂ and R₃ is trifluoromethyl, not more than one of R₂ and R₃ is phenoxy, and not more than one of R₂ and R₃ is benzyloxy.

The R₀, R₁, R₂, R₃, and R₄ positions of formula V′ above may be as defined for the R₀, R, R₂, R₃, and R₄ positions in the formulas of U.S. Pat. No. 5,354,772 in column 2, line 39, through column 9, line 32, and in the formulas of U.S. Pat. No. 4,739,073 in column 2, line 21, through column 8, line 37. Definitions that define those R groups are as further detailed in that Patent, which also provides reactions for the introduction of the various groups into formula V′.

Preferably, R₀ of formula V′ is —CH(CH₃)₂ while R₁, R₂, and R₃ are as defined in the formula. Alternatively, R₁ is a phenyl, optionally substituted with fluorine, chlorine, bromine, iodine or hydroxyl while R₀, R₂, and R₃ are as defined in formula V′; the substitution is preferably fluorine, and preferably at the para position relative to the bond linking the phenyl group to the rest of the molecule. Alternatively, R₂ is hydrogen R₀, R₁, and R₃ are as defined for formula V′; or R₃ is hydrogen while R₀, R₁, and R₂ are as defined for formula V′.

In yet another aspect, the invention provides non-statin compounds represented by the following formula VI.

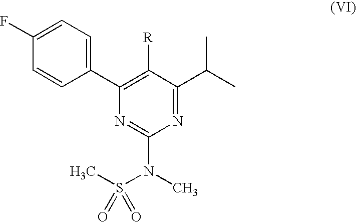

wherein R is as defined above for formula I.

The invention also provides derivative compounds based upon the core structure as depicted above and as represented by the following formula

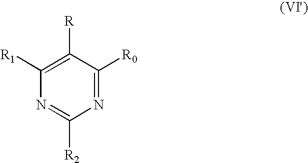

wherein R is as defined above for formula I, R₂ is the same as R, or R₂ is —N(CH₃)SO₂CH₃, as found in rosuvastatin, or an amide linkage (as in —N(CH₃)COCH₃ or —NHCOCH₃) or a urethane linkage (as in —N(CH₃)COOCH₃, or —NHCOOCH₃); and wherein one of R₁ and R₀ is

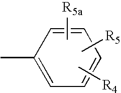

and the other is primary or secondary C₁₋₆ alkyl not containing an asymmetric carbon atom, C₃₋₆ cycloalkyl, phenyl, optionally substituted with fluorine, chlorine, bromine, iodine, CF₃, or hydroxyl, or phenyl-(CH₂)ₘ-, wherein

R₄ is hydrogen, C₁₋₃ alkyl, n-butyl, i-butyl, t-butyl, C₁₋₃ alkoxy, n-butoxy, i-butoxy, trifluoromethyl, fluoro, chloro, bromo, iodo, phenoxy or benzyloxy,

R₅ is hydrogen, C1-3 alkyl, C1-3 alkoxy, trifluoromethyl, fluoro, chloro, bromo, iodo, phenoxy or benzyloxy,

R₅ₐ is hydrogen, C1-2 alkyl, C1-2 alkoxy, fluoro or chloro, bromo, iodo, and

m is 1, 2 or 3, with the provisos that both R5 and R5a must be hydrogen when R4 is hydrogen, R5a must be hydrogen when R5 is hydrogen, not more than one of R4 and R5 is trifluoromethyl, not more than one of R4 and R5 is phenoxy, and not more than one of R4 and R5 is benzyloxy.

Alternatively, R₀ is isopropyl, isobutyl, isopentyl, as well as the sec-, normal, tert- or neo- forms thereof, such as n-butyl, and t-butyl; cyclopropyl, cyclobutyl, cyclopentyl or cyclohexyl; F; Br; Cl; I; trifluoromethyl or —OR′ wherein R′ is alkyl or aryl of C1-C10 and R₁ is as described above.

In a further aspect, the invention provides non-statin compounds represented by the following formula VII.

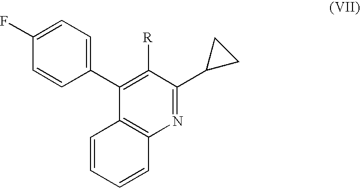

wherein R is as defined above for formula I.

The invention also provides derivative compounds based upon the core structure as depicted above and as represented by the following formula

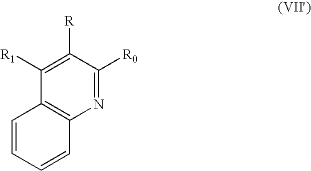

wherein R is as defined above for formula I; and wherein one of R₁ and R₀ is

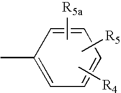

and the other is primary or secondary C₁₋₆ alkyl not containing an asymmetric carbon atom, C₃₋₆ cycloalkyl, phenyl, optionally substituted with fluorine, chlorine, bromine, iodine, CF₃, or hydroxyl, or phenyl-(CH₂)ₘ-; or wherein

R4 is hydrogen, C1-3 alkyl, n-butyl, i-butyl, t-butyl, C1-3 alkoxy, n-butoxy, i-btitoxy, trifluoromethyl, fluoro, chloro, bromo, iodo, phenoxy or benzyloxy,

R5 is hydrogen, C1-3 alkyl, C1-3 alkoxy, trifluoromethyl, fluoro, chloro, bromo, iodo, phenoxy or benzyloxy,

R5a is hydrogen, C1-2 alkyl, C1-2 alkoxy, fluoro or chloro, bromo, iodo, and

m is 1, 2 or 3, with the provisos that both R5 and R5a must be hydrogen when R4 is hydrogen, R5a must be hydrogen when R5 is hydrogen, not more than one of R4 and R5 is trifluoromethyl, not more than one of R4 and R5 is phenoxy, and not more than one of R4 and R5 is benzyloxy.

Alternatively, R₀ is isopropyl, isobutyl, isopentyl, as well as the sec-, normal, tert- or neo- forms thereof, such as n-butyl, and t-butyl; cyclopropyl, cyclobutyl, cyclopentyl or cyclohexyl; F; Br; Cl; I; trifluoromethyl or —OR′ wherein R′ is alkyl or aryl of C1-C10 and R₁ is as described above.

In a further aspect, the invention provides non-statin compounds represented by the following formula VIII.

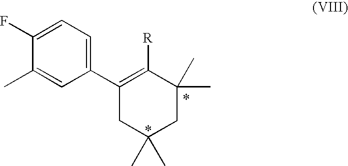

wherein R is as defined above for formula I.

The invention also provides derivative compounds based upon the core structure as depicted above and as represented by the following formula

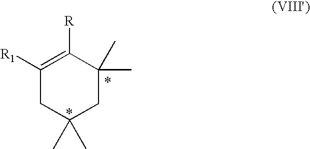

wherein R is as defined above for formula I; and wherein R₁ is

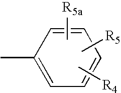

wherein

R₄ is hydrogen, C₁₋₃ alkyl, n-butyl, i-butyl, t-butyl, C₁₋₃ alkoxy, n-butoxy, i-butoxy, trifluoromethyl, fluoro, chloro, bromo, iodo, phenoxy or benzyloxy,

R5 is hydrogen, C1-3 alkyl, C1-3 alkoxy, trifluoromethyl, fluoro, chloro, bromo, iodo, phenoxy or benzyloxy,

R5a is hydrogen, C1-2 alkyl, C1-2 alkoxy, fluoro or chloro, bromo, iodo, and

m is 1, 2 or 3, with the provisos that both R5 and R5a must be hydrogen when R4 is hydrogen, R5a must be hydrogen when R5 is hydrogen, not more than one of R4 and R5 is trifluoromethyl, not more than one of R4 and R5 is phenoxy, and not more than one of R4 and R5 is benzyloxy.

In a further aspect, the invention provides non-statin compounds represented by the following formula IX.

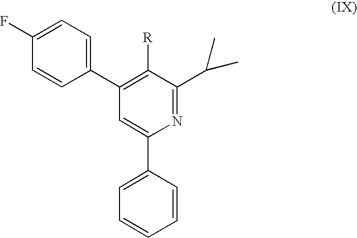

wherein R is as defined above for formula I.

The invention also provides derivative compounds based upon the core structure as depicted above and as represented by the following formula

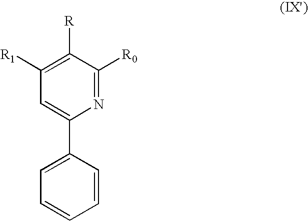

wherein R is as defined above for formula I; and wherein one of R₁ and R₀ is

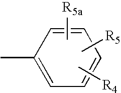

and the other is primary or secondary C₁₋₆ alkyl not containing an asymmetric carbon atom, C₃₋₆ cycloalkyl, phenyl, optionally substituted with fluorine, chlorine, bromine, iodine, CF₃, or hydroxyl, or phenyl-(CH₂)ₘ-, wherein

R₄ is hydrogen, C₁₋₃ alkyl, n-butyl, i-butyl, t-butyl, C₁₋₃ alkoxy, n-butoxy, i-butoxy, trifluoromethyl, fluoro, chloro, bromo, iodo, phenoxy or benzyloxy,

R5 is hydrogen, C1-3 alkyl, C1-3 alkoxy, trifluoromethyl, fluoro, chloro, bromo, iodo, phenoxy or benzyloxy,

R5a is hydrogen, C1-2 alkyl, C1-2 alkoxy, fluoro or chloro, bromo, iodo, and

m is 1, 2 or 3, with the provisos that both R5 and R5a must be hydrogen when R4 is hydrogen, R5a must be hydrogen when R5 is hydrogen, not more than one of R4 and R5 is trifluoromethyl, not more than one of R4 and R5 is phenoxy, and not more than one of R4 and R5 is benzyloxy.

Alternatively, R₀ is isopropyl, isobutyl, isopentyl, as well as the sec-, normal, tert- or neo- forms thereof, such as n-butyl, and t-butyl; cyclopropyl, cyclobutyl, cyclopentyl or cyclohexyl; F; Br; Cl; I; trifluoromethyl or —OR′ wherein R′ is alkyl or aryl of C1-C10 and R₁ is as described above.

In a further aspect, the invention provides non-statin compounds represented by the following formula X.

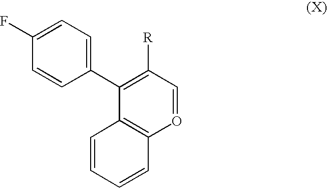

wherein R is as defined above for formula I.

The invention also provides derivative compounds based upon the core structure as depicted above and as represented by the following formula

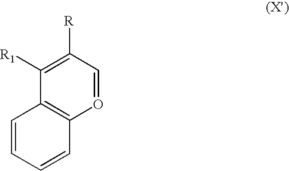

wherein R is as defined above for formula I; and wherein R₁ is

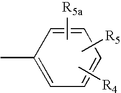

wherein

R4 is hydrogen, C1-3 alkyl, n-butyl, i-butyl, t-butyl, C1-3 alkoxy, n-butoxy, i-butoxy, trifluoromethyl, fluoro, chloro, bromo, iodo, phenoxy or benzyloxy,

R5 is hydrogen, C1-3 alkyl, C1-3 alkoxy, trifluoromethyl, fluoro, chloro, bromo, iodo, phenoxy or benzyloxy,

R5a is hydrogen, C1-2 alkyl, C1-2 alkoxy, fluoro or chloro, bromo, iodo, and

m is 1, 2 or 3, with the provisos that both R5 and R5a must be hydrogen when R4 is hydrogen, R5a must be hydrogen when R5 is hydrogen, not more than one of R4 and R5 is trifluoromethyl, not more than one of R4 and R5 is phenoxy, and not more than one of R4 and R5 is benzyloxy.

The invention also provides prodrug forms of the above described non-statin compounds, wherein the prodrug is metabolized in vivo to produce a derivative as set forth above. Indeed, some of the above described derivatives may be a prodrug for another derivative or active compound. The invention further provides for the optical isomers of the non-statin compounds disclosed herein, especially those resulting from the chiral carbon atoms in the molecule. In additional embodiments of the invention, mixtures of enantiomers and/or diastereoisomers, resulting from a single preparative step, combination, or interconversion are provided.

Methods of Synthesis

General methods for the preparation of non-statin compounds as disclosed herein may be derived from known reactions in the field. See for example Clive et al, J. Am. Chem 122:3018-3028 (1990) and Beck et al., J. Med. Chem. 33:52-60 (1990). A person of skill in the art can appropriately modify the known reactions by the use of appropriate reagents and conditions, for the introduction of the various moieties found in the formulae as provided herein.

Methods relating to the compounds of formulae I, I′, I″, I′″, I″″, I′″″, and II as derivatives of simvastatin, pravastatin, are provided in WO 89/08094. Methods relating to the compounds of formulae IV and IV′ as derivatives of fluvastatin are provided in U.S. Pat. No. 5,354,772. Methods relating to the compounds of formulae V and V′ as derivatives of cerivastatin are provided in U.S. Pat. Nos. 5,006,530, 5,169,857, and 5,177,080. Methods relating to the compounds of formulae VI and VI′ as derivatives of rosuvastatin are provided in U.S. Pat. No. 5,260,440. Methods relating to the compounds of formulae VII and VII′ as derivatives of pitavastatin are provided in U.S. Pat. Nos. 5,102,888, 5,854,259, 5,185,328, 5,872,130, and 5,856,336. Methods relating to the compounds of formulae VIII and VIII′ as derivatives of dalvastatin are provided in U.S. Pat. No. 4,863,957. Methods relating to the compounds of formulae IX and IX′ as derivatives of glenvastatin are provided in U.S. Pat. No. 4,925,852. Methods relating to the compounds of formulae X and X′ as derivatives of bervastatin are provided in U.S. Pat. Nos. 5,183,924 and 5,082,859.

Methods to synthesize atorvastatin are known in the field. (See Atorvastatin, AN HMG-CoA Reductase Inhibitor and Effective lipid-regulating agent. Part III Syntheses of [2H5]-, [13C8] and [13C7, 15N] Atorvastatin and their application in metabolic and pharmacokinetic studies'. J. Labelled Cpd. Radiopharm. 42, 135-145 (1999); Atorvastatin, AN HMG-CoA Redactase Inhibitor and Efficient Lipid-Regulatin Agent Part I. Synthesis of ring-labled [14C] atorvastatin. J. Labelled Cpd. Radiopharm. 42, 121-127 (1999); and Atorvastatin, AN HMG-CoA Redactase Inhibitor and Efficient Lipid-Regulatin Agent Part II. Synthesis of Side-Chain-Labled [14C] atorvastatin. J. Labelled Cpd. Radiopharm. 42, 129-133 (1999)).

Methods of Use

In a further aspect of the invention, methods for the use of the above described non-statin compounds, as well as compositions, are provided. These methods include uses of the invention's derivative compounds to provide the effects of the statin molecules not related to, or correlated with, the inhibition of HMG-CoA reductase activity. The methods of the invention also include those where it is unclear whether the effects of the non-statin compounds of the invention are also provided by the inhibition of HMG-CoA reductase activity mediated by statin molecules. Because the statins have been extensively tested in human subjects, the beneficial effects not related to, or correlated with, the inhibition of HMG-CoA reductase activity have been identified and documented. Exemplary effects include, but are not limited to, the treatment of cerebral accident (cerebrovascular accident or CVA), including stroke; inflammation; multiple sclerosis; angiogenesis; bone formation/bone growth; immune system stimulation; acute coronary syndromes; and cardiovascular disease, including the reduction of post-myocardial infarction trauma. The non-statin compounds of the invention may also be used in the prevention of such conditions, particularly in the cases of CVA such as in subjects that suffered one or more strokes or in subjects that have been diagnosed by medical personnel as at risk for a CVA, such as a stroke. Use of the derivatives in the prevention of a CVA, such as a stroke, may also be viewed as reducing the probability of a subject from having a CVA.

The methods of the invention may comprise the administration of one or a combination of more than one non-statin compound as disclosed herein, optionally in combination with one or more other active agents for the treatment of a disease or unwanted condition as disclosed herein. The non-statin compounds are preferably administered in an effective amount such that an effect equal to or greater than that seen with the corresponding statin compound is produced in a subject. The subject is preferably human, and repeated administration over time is within the scope of the present invention.

The invention also provides methods for determining the level of activity of a non-statin compound as disclosed herein in the treatment of a disease or unwanted condition as described herein. Such methods include the administration of a non-statin compound to a subject followed by determination of the level of activity of said derivative in comparison to a subject who has not been administered said derivative or to a subject that has been administered a different amount or concentration of said derivative. These methods may be practiced repeatedly, with a variety of amounts or concentrations of the derivative to determine the level of activity over a range of conditions. The methods may also be used to determine that the level of activity is undetectable.

An exemplary method of determining the level of activity of a non-statin compound may comprise

a) administering a non-statin compound as disclosed herein to a subject;

b) determining the level of efficacy against a disease or unwanted condition as disclosed herein in comparison to a subject (or group of subjects) that has not been administered said derivative or that has been administered a different amount of said derivative or administered said derivative under different administration protocols (such as, but not limited to, frequency of administration or amount of derivative administered).

The comparison may also be made between different non-statin compounds to determine their relative levels of activity. The subjects are animals, preferably human, and may be those that are part of a clinical or pre-clinical trial or test of one or more non-statin compounds. The determination of the level of activity can be made in a variety of ways as would be known to the skilled practitioner for the diseases and unwanted conditions disclosed herein.

A non-statin compound of the invention may be administered to a subject upon determination of the subject as having a disease or unwanted condition that would benefit by treatment with said derivative. The determination may be made by medical or clinical personnel as part of a diagnosis of a disease or condition in a subject. Non-limiting examples include determination of a risk of a CVA (such as stroke) or the effects of cardiovascular disease.

In the case of a CVA, such as stroke, and in one embodiment of the invention, a stain derivative of the invention may be used following an initial stroke to decrease the frequency and/or severity or damage, such as from brain infarction, that result from subsequent strokes. The invention thus provides methods for decreasing the damage resulting from stroke (post stroke trauma).

In the case of cardiovascular disease, and in one embodiment of the invention, a stain derivative of the invention may be used following an initial heart attack to decrease the frequency and/or severity or damage, such as from myocardial (heart) infarction (commonly referred to as heart attack), that result from subsequent heart attack(s). The invention thus provides for decreasing the frequency of and/or damage from post myocardial trauma.

The non-statin compounds of the invention may also be used to treat conditions such as inflammation and multiple sclerosis. Statin compounds have been identified as having anti-inflammatory activity as well as being effective against multiple sclerosis in animal models of multiple sclerosis.

Preferably, the compounds described herein are used in the treatment of PDE6-related conditions. Diseases that may be treated with the compounds described herein include, but are not limited to, cerebral accident (or cerebrovascular accident, including stroke), inflammation (including inflammation due to autoimmune diseases), multiple sclerosis, blood vessel growth (angiogenesis), bone formation/bone growth, immune system stimulation, acute coronary syndromes (including myocardial infarction, non-Q-wave myocardial infarction and unstable angina), and cardiovascular disease.

All the known statins and the non-statin compounds described herein may be used to treat PDE6-related conditions. In certain embodiments, the PDE6-related conditions are treated with non-statin compounds that lack the moiety that interacts with HMG CoA reductase. In other embodiments, the PDE6-related conditions are treated with the known statins, for example, lovastatin, simvastatin, pravastatin, fluvastatin, atorvastatin, rosuvastatin, pitavastatin, dalvastatin, glenvastatin, bervastatin, cerivastatin, and carvastatin.

The term “PDE6-related condition” as used herein refers to a condition in which directly or indirectly modulating the activity and/or production of a PDE6 molecule, respectively, is desirable. This modulation includes modulation of one or more molecules in the upstream or downstream sigrialing cascades of PDE6. For example, a PDE6-related condition may involve over-production or unwanted production of one or more prenylated PDE6 subunits, such as PDE6D, prenylated PDE6a or PDE6p, or other chemical messengers of cell signaling pathways associated with phototransduction (including responses to and expression of PDE6 alpha and PDE6 beta).

The PDE6-modulating agents of the invention can be administered to a mammalian subject to treat a disorder by modulating the binding of PDE6D to prenylated GTPases, thereby modulating GTPase-dependent signal transduction pathways. The disruption of GTPase-dependent pathways contributes to a variety of medical conditions, such as vascular hyperplasia, thrombin-induced cell death, the pathogenesis and progression of bladder cancer, chronic inflammatory disease, endothelial dysfunction in cardiovascular disease, cardiac hypertrophy, a change in cerebral blood flow to ischemic regions of the brain, phagocytosis of amloid-beta fibrils in Alzheimer's disease patients, immunodeficiency disorders and increased free radical production in aortic vascular smooth muscle cells.

PDE6-related conditions can include neurodegenerative diseases, including ischemic stroke, basal ganglia or Parkinson's disease, epilepsy or brains or spinal cord ischemia or trauma; Alzheimer's disease, dementia, diabetic peripheral neuropathy, multiple sclerosis, amyotrophic lateral sclerosis, traumatic brain injury, spinal cord injury, Huntington's disease, heart failure (e.g. congestive heart failure, acute heart failure, cardiac hypertrophy, etc.) or renal diseases.

PDE6-related conditions can include visual impairment disorders, including macular degeneration, amblyopia, Blepharitis, Bietti's Crystalline Dystrophy, comeal disease, diabetic eye disease, glaucoma, histoplasmosis, and retinitis pigmentosa. PDE6-related conditions can include cardiovascular-related conditions, including atherosclerosis, myocardial infarction, congestive heart failure, ischemic-reperfusion injury and other vascular inflammatory conditions. PDE6-related conditions can also include proliferative disorders, including cancers, e.g., leukemia, melanoma, Non-Hodgkins Lymphoma, as well as bladder, breast, colon, endometrial, head and neck, lung, ovarian, prostate and rectal cancers.

PDE6-related conditions can also include neurological deficits that develop from a stroke-induced impairment of blood flow to the brain regardless of cause. Potential causes include, but are not limited to, thrombosis, hemorrhage and embolism. Thrombus, embolus, and systemic hypotension are among the most common causes of cerebral ischemric episodes. Other injuries may be caused by hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from a tumor, or other blood loss.

In one embodiment, the PDE6-modulating agent modulates the activity small GTP binding protein Rho in its role in cell proliferation. It has been reported that Rho proteins are more abundant in tumor bladders than in non-tumor bladders and upregulated in ovarian carcinomas (Kamai T, et al., Clin. Cancer Res. Jul;9(7):2632-41 (2003) and Horiuchi A, et al., Lab Invest. 2003 Jun;83(6): 861-70 (2003)).

A disruption in Rho GTP binding activity has been shown to have the neuroprotective effect of increasing cerebral blood flow to ischemic regions of the brain (Laufs U, et al., J. Clin. Invest. 106(1):15-24 (2000)). The study demonstrated that under absent or decreased rho-dependent actin cytoskeleton stress fiber formation, eNOS was upregulated and the severity of cerebral ischemia was decreased. An embodiment of the invention provides for the treatment of ischemic stroke by modulation of Rho by a compound of the invention.

Researchers have reported that the cardiac hypertrophy, which requires intracellular oxidation may be reduced by statin-induced inhibition of post-translational modification of the small G proteins of the Rho family (Takemoto M, et al., J. Clin. Invest. 108(10):1429-37 (2001)). Takemoto M, et al. observed that an inhibition of the Rho isoprenylation produced an intracellular antioxidant effect and inhibit cardiac hypertrophy. One embodiment of the invention provides for the treatment of cardiac hypertrophy by modulation of Rho by a compound of the invention.

In another embodiment, the PDE6-modulating agent modulates the activity of the small GTP binding protein Rac in its role in Alzheimer's disease. Rac has been observed to participate in the phagocytosis of amyloid-beta fibrils from extracellular senile plaques. (Kitamura Y, et al, J. Pharmacol. Sci. 92(2): 115-23 2003)).

The present invention provides methods, pharmaceutical compositions, and kits for the treatment of subjects. As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like.

The term “treating” as used herein includes achieving a therapeutic benefit and/or a prophylactic benefit. By therapeutic benefit is meant eradication or amelioration of the underlying disorder being treated. For example, in a cancer patient, therapeutic benefit includes eradication or amelioration of the underlying cancer. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For example, a non-statin compound may provide a therapeutic benefit not only when Alzheimer's disease is eradicated, but also when an improvement is observed in the patient with respect to other disorders or discomforts that accompany Alzheimer's, like dementia. Similarly, non-statin compounds of the present invention can provide therapeutic benefit in ameliorating other symptoms associated with PDE6-related conditions, e.g., inflammatory, autoimmune, cancerous, impaired vision and/or neurodegenerative conditions, including redness, rashes, swelling, itching, irritation, dryness, scaling, flaking, pain, temperature increase, loss of normal function, and the like.

For prophylactic benefit, a composition of the invention may be administered to a patient at risk of developing a condition, for example a PDE6-related condition, or to a patient reporting one or more of the physiological symptoms of such conditions, even though a diagnosis of the condition may not have been made.

Another aspect of the invention relates to the method of using statins to treat a PDE6-related condition. The PDE6-related conditions treated include conditions in which direct or indirect modulation of the activity and/or production of PDE6, preferably PDE6D molecule, is desirable. This modulation includes modulation of one or more molecules in the upstream or downstream signaling cascades of PDE6 and/or PDE6D. For example, a PDE6-related condition may involve over-production or unwanted production of PDE6D subunits. When statins are used it is preferred that the conditions treated have a minimal HMG CoA reductase role. Preferably, the administration of statins produces a beneficial effect by preferably modulating PDE6 and/or PDE6D. Due to the properties of statins, an effect on HMG CoA reducatase activity would be anticipated, but in some embodiments, it is not this activity that produces a beneficial effect in the condition being treated. In certain embodiments, the effect of statins in the condition being treated or in the method in which it is employed is not reversed by the addition of farnesyl pyrophosphate, geraylgeranyl pyrophosphate, and/or mevalonate.

Formulations, Routes of Administration, and Effective Doses

The non-statin compounds are preferably used to prepare a medicament, such as by formulation into pharmaceutical compositions for administration to a subject using techniques generally known in the art. A summary of such pharmaceutical compositions may be found, for example, in Remington's Pharmaceutical Sciences, Mack Publishing Co., Easton, Pa. The compounds of the invention can be used singly or as components of mixtures. Preferred forms of the compounds are those for systemic administration as well as those for topical or transdermal administration. Formulations designed for sustained and/or delayed release are also with the scope of the invention.

Such pharmaceutical compositions can be used to treat PDE6-related conditions, as described in detail above, or suitable conditions. If necessary or desirable, the non-statin compounds may be administered in combination with other therapeutic agents. The choice of therapeutic agents that can be co-administered with the compositions of the invention will depend, in part, on the condition being treated.

The non-statin compounds may be administered per se or in the form of a pharmaceutical composition wherein the active compound(s) is in an admixture or mixture with one or more pharmaceutically acceptable carriers, excipients or diluents. Pharmaceutical compositions for use in accordance with the present invention may be formulated in conventional manner using one or more physiologically acceptable carriers compromising excipients and auxiliaries, which facilitate processing of the active compounds into preparations that can be used pharmaceutically. Proper. formulation is dependent upon the route of administration chosen. The non-statin compounds useful in the present invention can be delivered to the patient using a number of routes or modes of administration, including oral, buccal, topical, rectal, transdermal, transmucosal, subcutaneous, intravenous, and intramuscular applications, as well as by inhalation.

Methods for the preparation of compositions comprising the compounds of the invention include formulating the non-statin compounds with one or more inert, pharmaceutically acceptable carriers to form either a solid or liquid. Solid compositions include, but are not limited to, powders, tablets, dispersible granules, capsules, cachets, and suppositories. Liquid compositions include solutions, emulsions, or a solution containing liposomes, micelles, or nanoparticles comprising a compound as disclosed herein.

Compounds of this invention may also be integrated into foodstuffs, e.g, cream cheese, butter, salad dressing, or ice cream to facilitate solubilization, administration, and/or compliance in certain patient populations.

The compounds of the invention may be labeled isotopically (e.g. with a radioisotope) or by another other means, including, but not limited to, the use of chromophores or fluorescent moieties, bioluminescent labels, or chemiluminescent labels. The compositions may be in conventional forms, either as liquid solutions or suspensions, solid forms suitable for solution.or suspension in a liquid prior to use, or as emulsions. Suitable excipients or carriers are, for example, water, saline, dextrose, glycerol, alcohols, aloe vera gel, allantoin, glycerin, vitamin A and E oils, mineral oil, propylene glycol, PPG-2 myristyl propionate, and the like. Of course, these compositions may also contain minor amounts of nontoxic, auxiliary substances, such as wetting or emulsifying agents, pH buffering agents, and so forth.

For oral administration, the compounds can be formulated readily by combining the active compound(s) with pharmaceutically acceptable carriers well known in the art. Such carriers enable the compounds of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The compounds may also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

Aqueous suspensions may contain a compound of this invention with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

For injection, the compounds of the present invention may be formulated in aqueous solutions, preferably in physiologically compatible buffers such as Hank's solution, Ringer's solution, or physiological saline buffer. Such compositions may also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton Pa.

The compounds may also be formulated for transmucosal administration, buccal administration, for administration by inhalation, for parental administration, for transdermal administration, and rectal administration.

In addition to the formulations described previously, the compounds may also be formulated as a depot preparation. Such long acting formulations may be administered by implantation or transcutaneous delivery (for example subcutaneously or intramuscularly), intramuscular injection or use of a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In some embodiments, pharmaceutical compositions comprising compounds of the present invention exert local and regional anti-inflammatory effects when administered topically or injected at or near particular sites of inflammation. For example, ocular allergic, inflammatory and/or autoimmune conditions can be effectively treated with ophthalmic solutions, suspensions, ointments or inserts comprising one or more compounds of the present invention. Allergic, inflammatory and/or autoimmune conditions of the ear can be effectively treated with otic solutions, suspensions, ointments or inserts comprising one or more compounds of the present invention. Allergic, inflammatory and/or autoimmune conditions of the skin and skin structures can be effectively treated with skin ointments comprising one or more compounds of the present invention in an oleaginous hydrocarbon base, an anhydrous absorption base, a water-in-oil absorption base, an oil-in-water water-removable base and/or a water-soluble base. Gastrointestinal allergic, inflammatory and/or autoimmune conditions can be effectively treated with orally- or rectally delivered solutions, suspensions, ointments, enemas and/or suppositories comprising one or more compounds of the present invention. Respiratory allergic, inflammatory and/or autoimmune conditions can be effectively treated with aerosol solutions, suspensions or dry powders comprising one or more compounds of the present invention.

For example, for treating inflammatory and/or autoimmune conditions, a cream comprising a compound of the invention may be topically applied to the affected site, for example, sites displaying red plaques or dry scales in psoriasis, or areas of irritation and dryness in dermatitis. As another example, for treating inflammatory bowel disease, a suppository formulation of a compound disclosed herein can be used. In such embodiments, the active ingredient produces a benefit locally at or near the site of application, rather than systemically.

Direct topical application, e.g., of a viscous liquid, gel, jelly, cream, lotion, ointment, suppository, foam, or aerosol spray, may be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations may also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983.

In some preferred embodiments, the compounds of the present invention are delivered in soluble rather than suspension form, which allows for more rapid and quantitative absorption to the sites of action. In general, formulations such as jellies, creams, lotions, suppositories and ointments can provide an area with more extended exposure to the compounds of the present invention, while formulations in solution, e.g., sprays, provide more immediate, short-term exposure.

The formulations also may comprise suitable solid or gel phase carriers or excipients that increase penetration or help delivery of inhibitory compounds of this invention across the permeability barrier of the skin. Many of these penetration-enhancing compounds are known in the art of topical formulation. Examples of such carriers and excipients include humectants (e.g., urea), glycols (e.g., propylene glycol and polyethylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, ORGELASE, calcium carbonate, calcium phosphate, various sugars, starches, cellulose derivatives, gelatin, other polymers and water. In some embodiments, the pharmaceutical compositions will include one or more penetration enhancers such as water, methanol, ethanol, 2-propanol, dimethyl sulfoxide, decylmethyl sulfoxide, tetradecylmethyl sulfoxide, 2-pyrrolidone, N-methyl-2-pyrrolidone, N-(2-hydroxyethyl)pyrrolidone, laurocapram, acetone, dimethylacetamide, dimethylformamide, tetrahydrofurfuryl alcohol, L-α-amino acids, anionic surfactants, cationic surfactants, amphoteric surfactants, nonionic surfactants, fatty acids, fatty alcohols, clofibric acid amides, hexamethylene lauramide, proteolytic enzymes, α-bisabolol, d-limonene, urea, N,N-diethyl-m-toluamide, and the like.

In some embodiments, the pharmaceutical compositions will include one or more antimicrobial preservatives such as quaternary ammonium compounds, organic mercurials, p-hydroxy benzoates, aromatic alcohols, chlorobutanol, and the like.

Pharmaceutical compositions suitable for use in the present invention include compositions wherein the active ingredients are present in an effective amount. For example, the compounds of the present invention are present in an amount effective to achieve therapeutic and/or prophylactic benefit in a PDE6-related condition. The actual amount effective for a particular application will depend on the condition or conditions being treated, the condition of the subject, the formulation, and the route of administration, as well as other factors known to those of skill in the art. Determination of an effective amount of a compound described herein is well within the capabilities of those skilled in the art, in light of the disclosure herein, and will be determined using routine optimization techniques.

In therapeutic use, the compounds of the invention are administered to a subject at dosage levels of from about 0.05 mg/kg to about 10.0 mg/kg of body weight per day. For a human subject of approximately 70 kg, a dosage of from 40 mg to 600 mg per day may be used as a non-limiting example. The dosages of the non-statin compounds can be the same as, less than, or more than the corresponding doses for the statins. The dosages of the statins can be the same as, less than, or more than the doses used in the art. Such dosages, however, may be altered depending on a number of variables, not limited to the activity of the compound used, the condition to be treated, the mode of administration, the requirements of the individual subject, the severity of the condition being treated, and the judgment of the practitioner. The foregoing ranges are merely suggestive, as the number of variables in regard to an individual treatment regime is large, and considerable excursions from these recommended values are not uncommon.

The effective amount for use in humans can be determined from animal models. For example, a dose for humans can be formulated to achieve circulating, liver, topical and/or gastrointestinal concentrations that have been found to be effective in animals.

The effective amount when referring to an inhibitor of the invention will generally mean the dose ranges, modes of administration, formulations, etc., that have been recommended or approved by any of the various regulatory or advisory organizations in the medical or pharmaceutical arts (eg, FDA, AMA) or by the manufacturer or supplier.

In some embodiments, administration of compounds of the present invention may be intermittent, for example administration once every two days, every three days, every five days, once a week, once or twice a month, and the like. In some embodiments, the amount, forms, and/or amounts of the different forms may be varied at different times of administration.

Kits/Articles of Manufacture

For use in the therapeutic applications described herein, kits and articles of manufacture are also within the scope of the invention. Such kits can comprise a carrier, package, or container that is compartmentalized to receive one or more containers such as vials, tubes, and the like, each of the container(s) comprising one of the separate elements to be used in a method of the invention. Suitable containers include, for example, bottles, vials, syringes, and test tubes. The containers can be formed from a variety of materials such as glass or plastic.

For example, the container(s) can comprise one or more non-statin compounds of the invention, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a non-statin compound with an identifying description or label or instructions relating to its use in the methods of the present invention.

A kit of the invention typically may comprise one or more additional containers, each with one or more of various materials (such as reagents, optionally in concentrated form, and/or devices) desirable from a commercial and user standpoint for use of a non-statin compound of the invention. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

The terms “kit” and “article of manufacture” may be used as synonyms.

Having now generally described the invention, the same will be more readily understood through reference to the following examples which are provided by way of illustration, and are not intended to be limiting of the present invention, unless specified.

All publications and patent applications mentioned in this specification are herein incorporated by reference to the same extent as if each individual publication or patent application was specifically and individually indicated to be incorporated by reference.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A statin composition comprising a non-statin compound wherein said non-statin compound is a statin compound lacking a HMG CoA reductase moiety, wherein said HMG CoA reductase moiety interacts with an HMG CoA reductase enzyme.

2. The non-statin compound of claim 1 wherein said HMG CoA reductase moiety is a heptanoic carboxylic acid or a lactone ring.

3. The non-statin compound of claim 1 wherein said HMG CoA reductase moiety is at least one of the following groups 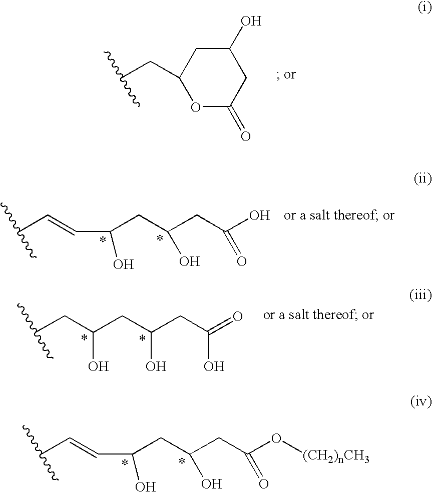
wherein n is an integer.

4. A pharmaceutical composition comprising the composition of claim 1 and a pharmaceutically acceptable excipient.

5. A method of treating a disease comprising administering to a subject in need thereof an effective amount of the composition of claim 1.

6. The method of claim 5 wherein said disease is a cerebrovascular accident.

7. The method of claim 6 wherein said cerebrovascular accident is stroke.

8. The method of claim 6 wherein said subject has previously suffered a stroke.

9. The method of claim 5 wherein said disease is a cardiovascular disease.

10. The method of claim 9 wherein said treatment of cardiovascular disease decreases the frequency and/or the severity or damage of myocardial infarction in said subject.

11. The method of claim 5 wherein said disease is inflammation or multiple sclerosis.

12. The method of claim 5 wherein said disease is an acute coronary syndrome.

13. The method of claim 12 wherein said syndrome is selected from myocardial infarction, non-Q-wave myocardial infarction and unstable angina.

14. A method of stimulating blood vessel growth, bone growth, and/or an immune system comprising administering to a subject in need thereof an effective amount of a compound of claim 1.

15. The method of claim 5 wherein said disease is a PDE6-related condition.

16. The method of claim 15 wherein said PDE6-related condition is at least one of a condition selected from neurodegenerative condition, visual impairment disorder, proliferative. disorders, and cardiovascular-related condition.

17. The method of claim 16 wherein said neurodegenerative condition is at least one condition selected from ischemic stroke, basal ganglia, Parkinson's disease, epilepsy, brain or spinal cord ischemia or trauma, Alzheimer's disease, dementia, diabetic peripheral neuropathy, multiple sclerosis, amyotrophic lateral sclerosis, traumatic brain injury, spinal cord injury, and Huntington's disease.

18. The method of claim 16 wherein said visual impairment disorder is at least one discorder selected from macular degeneration, amblyopia, Blepharitis, Bietti's Crystalline Dystrophy, comeal disease, diabetic eye disease, glaucoma, histoplasmosis, and retinitis pigmentosa.

19. The method of claim 16 wherein said cardiovascular-related condition is at least one condition selected from atherosclerosis, myocardial infarction, congestive heart failure, ischemic-reperfusion injury, and vascular inflammatory conditions.

20. The method of claim 16 wherein said proliferative disorder is at least one disorder selected from leukemia, melanoma, Non-Hodgkins Lymphoma, bladder cancer, breast cancer, colon cancer, endometrial cancer, head and neck cancer, lung cancer, ovarian cancer, prostate cancerand rectal cancer.

21. A method of treating a PDE6-realated condition comprising administering to a subject in need thereof an effective amount of a statin compound.

22. The method of claim 21 wherein said statin compound is at least one of a statin selected from the following lovastatin, simvastatin, pravastatin, fluvastatin, atorvastatin, rosuvastatin, pitavastatin, dalvastatin, glenvastatin, bervastatin, cerivastatin, and carvastatin.
